(19) United States
(12) Patent Application Publication
(10) Pub. No.: US 2025/0269131 A1
(43) Pub. Date: Aug. 28, 2025

(54) DEVICES AND METHODS FOR PERFORMING A TRACHEOSTOMY

(71) Applicants: Mohamad Chmayssani, Ponte Vedra Beach, FL (US); Michael Schaller, Louisville, CO (US)

(72) Inventors: Mohamad Chmayssani, Ponte Vedra Beach, FL (US); Michael Schaller, Louisville, CO (US)

(21) Appl. No.: 19/207,886

(22) Filed: May 14, 2025

Related U.S. Application Data
(63) Continuation of application No. PCT/US2023/079661, filed on Nov. 14, 2023.
(60) Provisional application No. 63/528,579, filed on Jul. 24, 2023.
(60) Provisional application No. 63/472,893, filed on Jun. 14, 2023.
(60) Provisional application No. 63/425,080, filed on Nov. 14, 2022.

(51) Int. Cl. A61M 16/04; A61B 1/267; A61M 25/01

(52) U.S. Cl. CPC A61M 16/0472; A61M 16/0488; A61B 1/2676; A61M 25/0102; A61M 2205/587

(57) ABSTRACT

A method for performing a tracheostomy may include positioning a locating tip of a guide proximate to or in contact with at least a portion of an anterior surface of a trachea, the guide and the locating tip having a guide lumen extending therethrough. A method may include creating a visual indicator within the trachea. A method may include inserting a needle through the guide lumen and into the trachea according to the visual indicator.

### CROSS-REFERENCE TO RELATED APPLICATIONS

This application claims the priority benefit of each of: U.S. Provisional Patent Application No. 63/425,080, filed Nov. 14, 2022, titled “DEVICES AND METHODS FOR PERFORMING A TRACHEOSTOMY”; U.S. Provisional Patent Application No. 63/472,893, filed Jun. 14, 2023, titled “DEVICES AND METHODS FOR PERFORMING A TRACHEOSTOMY”; and U.S. Provisional Patent Application No. 63/528,579, filed Jul. 24, 2023, titled “DEVICES AND METHODS FOR PERFORMING A TRACHEOSTOMY,” the entire contents of each of which are incorporated herein by reference for all purposes.

### TECHNICAL FIELD

This disclosure relates generally to the field of medicine, and more specifically to the field of airway management. Described herein are devices and methods for performing a tracheostomy.

### BACKGROUND

A tracheostomy is a procedure where a tube is placed into the trachea of a patient and can be performed for a variety of reasons such as obstructed airways, facilitation of mechanical ventilation, more efficient management of secretions, or any number of other indications. A method of performing such a procedure may include the following steps: 1) placement of a needle and cannula into the trachea, 2) removal of the needle, 3) insertion of a guidewire through the cannula, 4) removal of the cannula, 5) placement of a dilator or series of dilators to widen the entry hole, and 6) placement of a tracheostomy tube into the trachea. A challenging step in such a procedure is the initial placement of the needle and cannula into the trachea which forms the entry hole. This can be particularly difficult in obese patients and patients without clear anatomical landmarks.

### SUMMARY

The present disclosure is directed to devices and methods for performing a tracheostomy. In one application, the devices and methods are used to guide the placement of the needle and cannula into the trachea during a tracheostomy. The device may include a guide lumen, a locating tip, a tissue indenting feature, and a depth controlling stop. In one implementation, when the device is used, the tissue indenting feature provides a visual indication within the trachea of the midline position of the locating tip. The guide lumen then directs the needle and cannula to the locating tip and into the trachea. The depth controlling stop prevents the needle and cannula from going too far into the trachea.

In another implementation, the device may include a light source that creates an illuminated tissue which provides a visual indication within the trachea of the position of the locating tip.

In some cases, the device may be further configured to include a finger interface that enables the user to press the tissue indenting feature into the trachea and steadily hold the locating tip in place.

In some cases, the device may also include a probe with a blunt end that is inserted through the guide lumen and used as a tissue indenting feature.

In some aspects, the techniques described herein relate to a method of performing a tracheostomy procedure, including positioning a locating tip of a guide proximate to or in contact with at least a portion of an anterior surface of a trachea, the guide and the locating tip having a guide lumen extending therethrough; creating a visual indicator within the trachea; and inserting a needle through the guide lumen and into the trachea according to the visual indicator.

In some aspects, the techniques described herein relate to a device for performing a tracheostomy procedure, including: a guide defining a guide lumen and including; a locating tip having a tissue indenting feature, wherein the guide lumen extends through the locating tip; and a depth controlling stop for controlling a depth of insertion of a needle through the guide lumen and into a lumen of a trachea.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various implementations, with reference made to the accompanying drawings.

FIG. 1A illustrates a guide of an implementation of a device according to the present disclosure in an isometric view.

FIG. 1B illustrates an alternate isometric view of the device of FIG. 1A.

FIG. 2A illustrates the device of FIG. 1A including a needle and cannula in an exploded view.

FIG. 2B illustrates the device of FIG. 1A with the needle and cannula assembled.

FIG. 2C illustrates the device of FIG. 1A with the needle and cannula positioned in the guide.

FIG. 2D illustrates the device of FIG. 1A with the cannula positioned in the guide.

FIG. 3A illustrates the guide of the device of FIG. 1A over a trachea of a patient.

FIG. 3B illustrates the guide of FIG. 1A indenting the tissue.

FIG. 3C illustrates the guide of FIG. 1A with a needle and cannula inserted through the trachea.

FIG. 3D illustrates the device of FIG. 3C with the needle removed.

FIG. 4A illustrates an implementation of the device with a probe in an exploded view.

FIG. 4B illustrates the device of FIG. 4A with the guide and probe assembled.

FIG. 4C illustrates the device of FIG. 4A with the probe indenting the tissue.

FIG. 5A illustrates an isometric view of another implementation of a guide.

FIG. 5B illustrates an isometric view of another implementation of a guide.

FIG. 6 illustrates an implementation of a method of use of a tracheostomy device.

FIG. 7A illustrates an implementation of a device with a light source.

FIG. 7B illustrates the light source and guide of FIG. 7A connected.

FIG. 7C illustrates the light source of FIG. 7A causing tissue to be illuminated.

FIG. 8A illustrates an implementation of a device with an integrated light source in the guide.

FIG. 8B illustrates the light transmitted through the lumen of the device.

FIG. 8C illustrates the light transmitted through a light pipe.

FIG. 9A illustrates an alternative implementation of a guide with an integrated light source.

FIG. 9B illustrates the light transmitted through the guide.

FIG. 10A illustrates a device with a light source that can be connected to the needle.

FIG. 10B illustrates the light source connected to the needle.

FIG. 10C illustrates the needle inserted into the guide in a retracted position above a trachea.

FIG. 10D illustrates the needle advanced into the trachea.

FIG. 10E illustrates the needle remove and cannula in place.

FIG. 10F illustrates a guidewire inserted into the cannula.

FIG. 10G illustrates the cannula and guide removed with the guidewire in place.

FIG. 11A illustrates a guide with a sharp protrusion.

FIG. 11B illustrates the sharp protrusion inserted into the tracheal lumen.

FIG. 12A illustrates a stylet with a sharp protrusion inserted into the tracheal lumen.

FIG. 12B illustrates the needle and cannula guided over the stylet.

### DETAILED DESCRIPTION

Conventional and known tracheostomy devices include a variety of mechanisms and approaches to placing a tracheostomy tube. Various commercially available tracheostomy kits involve the dilation of a hole for the tracheostomy tube. The dilation occurs using one or more dilators over a guidewire to expand the hole that the guidewire is in. The location of the hole is therefore important to the success of the procedure and reduction of complications such as broken tracheal rings. The initial hole is created by placing a needle and cannula assembly through the anterior surface of the trachea and into the tracheal lumen. Medical staff are challenged to insert the needle assembly in the desired location which is often on the midline of the trachea between the second and third tracheal rings, because the trachea often lacks good visual indicators on its external surface. This may result in a puncture that is in a less desirable location or may require multiple punctures which damages surrounding tissue structures.

Further, limitations of some existing devices and methods include but are not limited to, insufficient user control and stability of the needle during insertion into the trachea, inaccurate placement of the cannula off the midline of the trachea, damage to tracheal rings or other tissue, and lack of depth control of the needle such that it may damage the backwall of the trachea.

Described herein are devices and methods which overcome the previously described challenges, among others, while providing substantial additional benefits related to the placement of a tracheostomy tube. Further, the described devices and methods can assist in the placement of the needle and cannula while overcoming some or all of the aforementioned limitations.

During use, the disclosed device may be generally used to create a visual indicator within the trachea of a patient and guide a needle assembly to the visually indicated location. In one aspect, the disclosed devices may use a physical indentation, a tissue illumination, or a sharp protrusion as a visual indicator. By first visualizing within the trachea (e.g., via a bronchoscope) the position of the locating tip of the guide, the guide lumen can then direct the needle, cannula, or other components to this location. In some implementations, the disclosed device may further include a depth limiting feature to control how far the needle is inserted into the trachea and prevent backwall injuries.

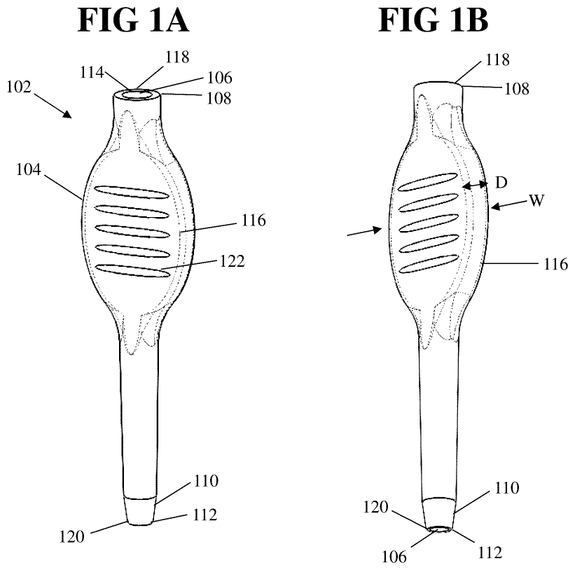

In FIGS. 1A-1B, a device (102) to perform a tracheostomy is shown. The device (102) includes a guide (104) component. The guide (104) defines a guide lumen (106) that runs a longitudinal length of the guide (104) from a guide proximal end (118) (e.g., cannula or needle insertion end) to the locating tip (110) or guide distal end (120). At the guide distal end (120) of the guide (104), there is a tissue indenting feature (112). The guide (104) also includes a depth controlling stop (108) and a cannula engagement feature (114) at the proximal end. The guide (104) further includes a finger interface (116) along its length. In FIG. 1B, the guide (104) is shown from a different isometric view that shows the guide lumen (106) and the tissue indenting feature (112) at the distal end of the guide (104).

The locating tip (110) functions to position the needle at the desired insertion point on the trachea based upon one or more visual indicators created by the device (102) within the trachea (304). The locating tip (110) may include a full or partial lumen for guiding the needle to provide the guided access site for the needle (202) as it is placed against, proximate to, or in contact with the anterior surface (302) of the trachea (304). The locating tip (110) may include an atraumatic indenting feature (112) for pressing against a trachea (304) of the patient. The tissue indenting feature (112) can be a blunt surface that does not puncture the tissue but presses on the tissue to cause an indentation. The indenting feature (112) may be sized and/or shaped to indent a portion of the trachea, for example between the second and third tracheal rings. For example, a diameter of the indenting feature (112) may be from and including about 0.030 inches (0.76 mm) to and including about 0.500 inches (12.7 mm); or from and including about 0.080 inches (2.03 mm) to and including about 0.200 inches (5.08 mm); or about 0.125 inches (3.175 mm), for example depending on a size of the patient. In some implementations, a material of the indenting feature (112) may be atraumatic such that the indenting feature (112) is formed of or comprises one or more of nylon, polycarbonate, silicone, stainless steel, or a combination thereof. In some implementations, the tissue indenting feature (112) can be tapered as shown to provide better visual clarity of the locating tip (110). A tapered tip also may beneficially provide greater feedback to the user about the location of the locating tip (110) relative to the trachea rings. Any number of other implementations of the tissue indenting feature (112) and locating tip (110) are contemplated and some will be discussed further herein.

In some embodiments, the locating tip (110) and the tissue indenting feature (112) are substantially the same component, located along at least a portion of the guide distal end (120). In some embodiments, a guide includes a locating tip (110) but does not include a tissue indenting feature (112), for example guides that include a light source or a probe for creating a visual indicator. In some embodiments, a guide includes a locating tip (110, a tissue indenting feature (112), and a light source.

The depth controlling stop (108), as shown in FIGS. 1A-1B, functions to engage or interact with a needle being extended through a lumen of the device (102) and prevent over penetration of the needle into a trachea of the patient. The depth controlling stop (108) of FIGS. 1A-1B is shown as a flat surface (but could also be an angled surface) at the guide proximal end (118) of the guide (104), but can also be any other detent, stop, or otherwise that prevents further movement in a direction.

The cannula engagement features (114), as shown in FIGS. 1A-1B, functions to engage or interact with a cannula being extended through the guide lumen (106). The depth controlling stop (108) of FIGS. 1A-1B is shown as a flat surface of a guide proximal end (118) of the guide (104), but can also be any other detent, stop, or otherwise that prevents further movement in a direction.

The finger interface (116) functions to be gripped or manipulated by a user during a tracheostomy. For example, finger interface (116) has a width W that is greater than a diameter D of the body of the guide (104). For example, the finger interface (116) may have a width from and including about 0.100 inches (2.54 mm) to and including about 2.0 inches (50.8 mm); from and including about 0.250 inches (6.35 mm) to and including about 1.0 inches (25.4 mm); or about 0.625 inches (15.88 mm), for example, depending on a size of hand of the user. In some implementations, the finger interface (116) may have a width W that is greater than a depth D, such that the finger interface (116) extends out from the guide (104) in a lateral direction. For example, a depth of the finger interface (116) may be from and including about 0.050 inches (1.27 mm) to and including about 1.000 inches (25.4); from and including about 0.100 inches (2.54 mm) to about 0.500 inches (12.7 mm); or about 0.200 inches (5.08 mm). In some implementations, the finger interface (116) may include one or more tactile or gripping features (122) to enhance gripping or grasping of the finger interface (116) by a user.

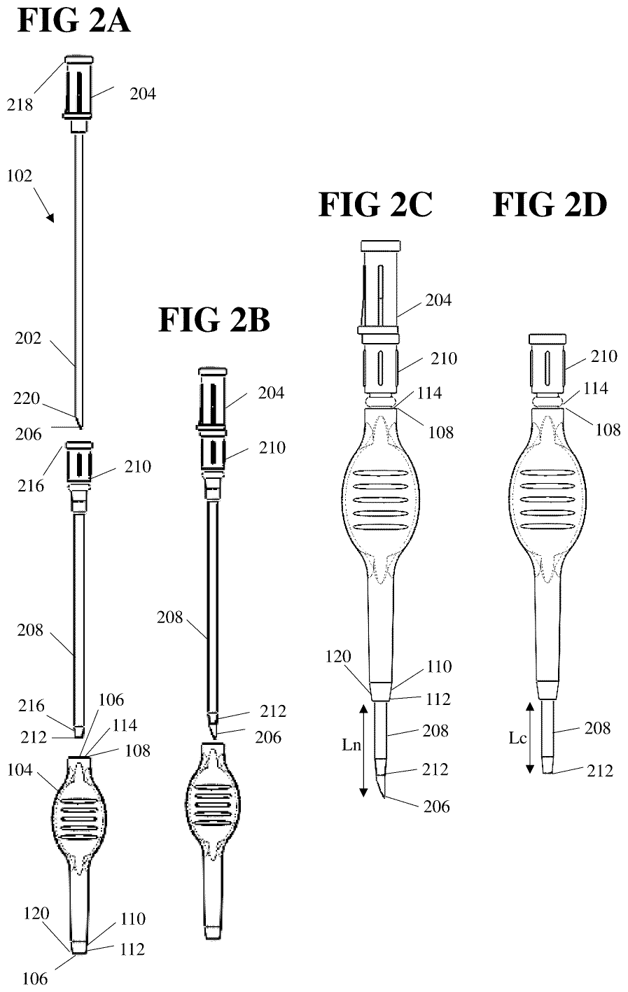

In FIGS. 2A-2D, the device (102) is shown with a guide (104), a needle (202) and a cannula (208). In FIG. 2A, the components are shown in an exploded pre-assembled view. The needle (202) has needle hub (204) at the needle proximal end (220) and needle tip (206) at the needle distal end (218). The cannula (208) has a cannula hub (210) at the cannula proximal end (214) and cannula tip (212) at the cannula distal end (216). In FIG. 2B, the needle (202) is shown assembled into the cannula (208) (i.e., needle is inserted through a lumen defined by the cannula (208)) with the needle tip (206) extending beyond the cannula tip (212). In FIG. 2C, the needle (202) and cannula (208) assembly are inserted into a guide lumen (106) defined by the guide (104). The cannula hub (210) is pressed up against or abutting the depth controlling stop (108) which limits how far the needle tip (206) and cannula tip (212) extend past the tissue indenting feature (112) defined by the needle length Ln and cannula length Lc. The depth controlling stop (108) is a hard stop defined by the flat surface on the guide proximal end (118) of the guide (104) that the cannula hub (210) engages or interacts with as the cannula (208) is advanced into the guide lumen (106). The cannula hub (210) also engages or interacts with the cannula engagement feature (114) to connect the cannula (208) to the guide (104). In FIG. 2D, the needle (202) is removed from the assembly. The cannula hub (210) remains connected to the cannula engagement feature (114) which maintains cannula length Lc as the needle (202) is removed.

The guide (104) can be formed of or comprise: a plastic, a metal such as stainless steel, or any other suitable material. In some implementations, the guide (104) can be formed of or comprise: a translucent material such as a clear plastic that provides better visualization during the procedure. The guide (104) is shown as a single component (monolithic construction) but, in other implementations, may include or be formed of multiple components that, when assembled together, create any of the guides described herein. For example, the guide (104) may be formed as a first side and a second side that snap together or are friction fit together to form the guide (104). Further, for example, the guide (104) may be formed of a first end and a second end such that the two ends snap together, are screwed to one another, etc. In another implementation, the guide (104) may be formed of a hypotube that forms the guide lumen (106) and a separate part that forms the body of the guide (104) including the finger interface (116). The overall length of the guide (104) can be from and including about 0.1 inches (2.54 mm) to and including about 8.0 inches (203.2 mm), or from and including about 1.0 inch (25.4 mm) to and including about 4.0 inches (101.6 mm), or about 2.5 inches (63.5 mm). The guide lumen (106) can be sized to accommodate the needle (202) and cannula (208) with a hole diameter from and including about 0.02 inches (0.51 mm) to and including about 0.25 inches (6.35 mm), or from and including about 0.06 inches (1.52 mm) to and including about 0.15 inches (3.81 mm), or about 0.10 inches (2.54 mm). A locating tip (110) is at the guide distal end (120) of the guide (104) which includes the guide lumen (106) extending therethrough.

The finger interface (116) can be an elliptical pad as shown with a width from and including about 0.2 inches (5.08 mm) to and including about 1.0 inch (25.4 mm) or about 0.6 inches (15.24 mm). The finger interface (116) can include grip features (122) such as ribs, embosses, or the like that provide a texture for the user, for example to enhance gripping of the guide (104). The depth controlling stop (108) can be a hard stop formed by the guide lumen (106) which is smaller than the cannula hub (210). As the cannula hub (210) moves toward the guide proximal end (118) of the guide (104), it is prevented from moving further since it cannot fit within the guide lumen (106) and therefore that limits the depth of the cannula hub (210). In other implementations, the depth controlling stop can interface with other elements of the cannula (208) or the needle (202). For example, the cannula hub (210) or needle hub (204) may have a protrusion that slides in a slot formed by the guide lumen (106) and includes one or more depth controlling stops (108). The guide proximal end (118) of the guide (104) also includes a cannula engagement feature (114) that interfaces with the cannula hub (210). In some implementations, the cannula engagement feature (114) can be a press fit interface with the cannula hub (210) that snaps the two components together. In other implementations, the cannula engagement feature (114) can include a threaded or bayonet connection or Luer lock such that guide (104) and cannula (208) are securely connected. The cannula engagement feature (114) can enable the cannula (208) to remain in place as the needle (202) is removed. In other implementations, the guide (104) does not include a cannula engagement feature (114). In such implementations, the cannula may be held in place by the user while the needle is removed.

The needle (202) may comprise or be manufactured in a manner consistent with existing tracheostomy needles such as a stainless-steel needle tip (206) with a plastic needle hub (204). Similarly, the cannula (208) may comprise or be manufactured in a manner consistent with existing tracheostomy cannulas such as a soft catheter tube and a plastic cannula hub (210). The needle length Ln may be appropriately sized to enter the trachea without touching the back wall of the trachea. The needle length Ln can be from and including about 0.2 inches (5.08 mm) to and including about 2.0 inches (50.8 mm), or from and including about 0.5 inches (12.7 mm) to and including about 1.5 inches (38.1 mm), or about 0.8 inches (20.32 mm). Similarly, the cannula length Lc can be appropriately sized so that the needle tip (206) extends past the cannula tip (212), and the cannula tip (212) fully enters the trachea. The cannula length Lc can be from and including about 0.1 inches (2.54 mm) to and including about 1.9 inches (48.26 mm), or from and including about 0.4 inches (10.16 mm) to and including about 1.4 inches (35.56 mm) or about 0.6 inches (15.24 mm).

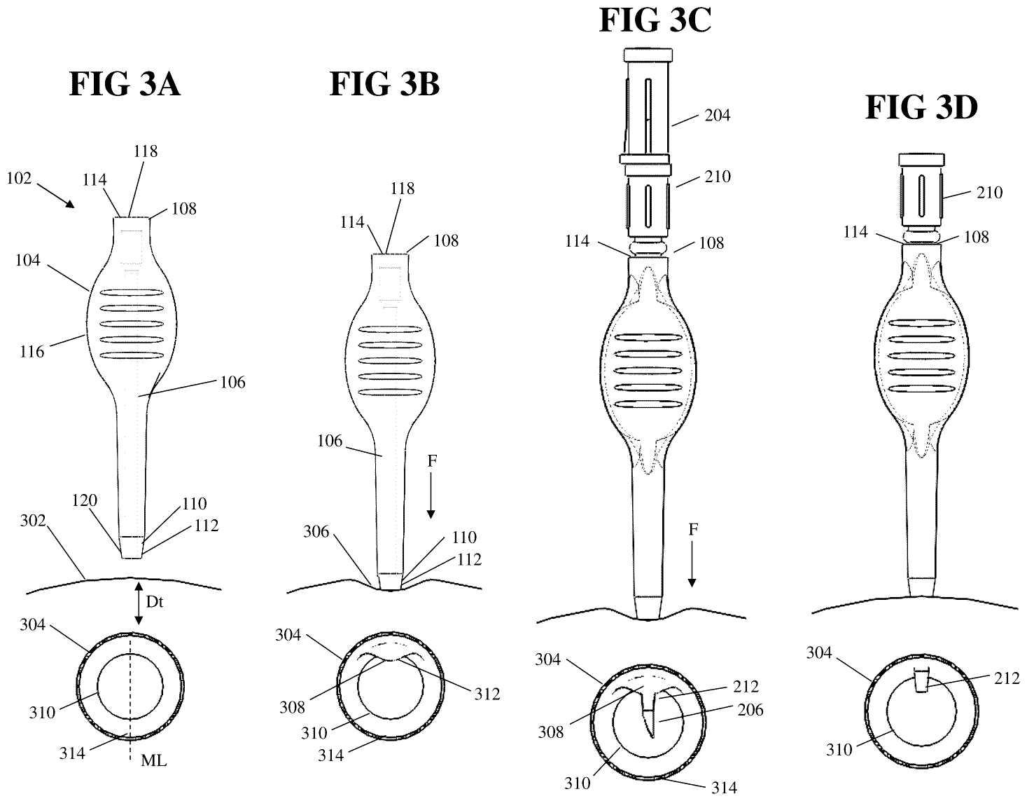

In FIGS. 3A-3D, the operation of the device (102) is shown in greater detail. In FIG. 3A, the guide (104) is shown above the anterior surface (302) of a trachea (304). There is a tracheal lumen (310) below the anterior surface (302) of the trachea (304) at a tissue depth Dt. The tracheal lumen (310) is represented as a generally cylindrical tube with a midline ML that runs down the medial plane of the trachea dividing the left and right sides. The internal view of the trachea (304) can be visualized by a bronchoscope or other similar visualization device that enables an intralumenal assessment of the tracheal lumen (310) such that the user can see the midline ML. In FIG. 3B, the tissue indenting feature (112) is pressed into the anterior surface (302). The user can use the finger interface (116) to grip the guide (104) with an index finger and thumb, or by any other suitable method. This enables the user to provide the downward force F of the guide (104) onto the anterior surface (302) and which causes an external indentation (306). Within the tracheal lumen (310), the tissue indenting feature (112) further creates a trachea indentation (308) which can be visualized by bronchoscope or other method. Ideal placement of the cannula (208) is often on the midline ML of the tracheal lumen (310). The user can observe the visual indicator (312) where the trachea indentation (308) occurs and determine if it is on the midline ML of the tracheal lumen (310) or not. Optionally, the user can move or adjust the guide (104) using the finger interface (116) to adjust the location of the tissue indenting feature (112). In some implementations, the user may desire the cannula (208) to be placed in a location other than the midline ML of the trachea. The visual indicator (312) of the trachea indentation (308) allows the user to test multiple locations on the anterior surface (302) and confirm the location within the tracheal lumen (310) before placement of the needle (202) and cannula (208). Additionally, the tissue indenting feature (112) can be used to determine the location of the locating tip (110) relative to the tracheal rings. Tracheostomy tubes are often ideally placed between tracheal rings, typically between the second and third tracheal ring however it can also be placed below the first ring. The tissue indenting feature (112) can allow the user to determine the location of the locating tip (110) relative to these rings by feeling for the valleys between the rings. The tracheal rings may also be observed by the visual indicator (312) of the trachea indentation (308).

In FIG. 3C, the needle (202) is inserted into the cannula (208), which is inserted into the guide lumen (106) that extends through the guide (104) and into the locating tip (110). The locating tip (110) ensures that the entry point for the needle tip (206) is accurate relative to the tissue indenting feature (112). The needle tip (206) and cannula tip (212) enter into the tracheal lumen (310). At the guide proximal end (118) of the guide (104), the cannula hub (210) interacts with the depth controlling stop (108) which limits how far the needle tip (206) extends past the tissue indenting feature (112) to the needle length Ln. In the implementations shown, the needle length Ln and cannula length Lc are longer than the tissue depth Dt such that needle tip (206) extends into the tracheal lumen (310) but is prevented from touching the backwall (314) of the tracheal lumen (310). The user can continue to grip the finger interface (116) with one hand while the other hand slowly moves the needle (202) and cannula (208) down. This provides the user with good control of the needle (202) placement during this part of the procedure. In some implementations, after confirming the location of the locating tip (110) by observing the visual indicator (312) of the trachea indentation (308), the user can reduce the downward force F on the guide (104) so that the trachea indentation (308) is reduce or eliminated. The user can maintain the locating tip (110) in the same location to ensure that the needle (202) enters at the desired position.

In FIG. 3D, the needle (202) is removed from the cannula (208) and thus the guide (104). The cannula hub (210) is interfaced with the cannula engagement feature (114) and therefore as the needle is removed, the cannula (208) remains in place relative to the guide (104) that is still held by the user. The cannula tip (212) likewise remains in place within the tracheal lumen (310). At this point, the remainder of the tracheostomy procedure may be performed by placing a guidewire through the cannula (208) and then removing the cannula (208). With the guidewire in place, a dilator or series of dilators can be advanced over the guidewire to enlarge the opening to the trachea. A tracheostomy tube can then be placed into the opening.

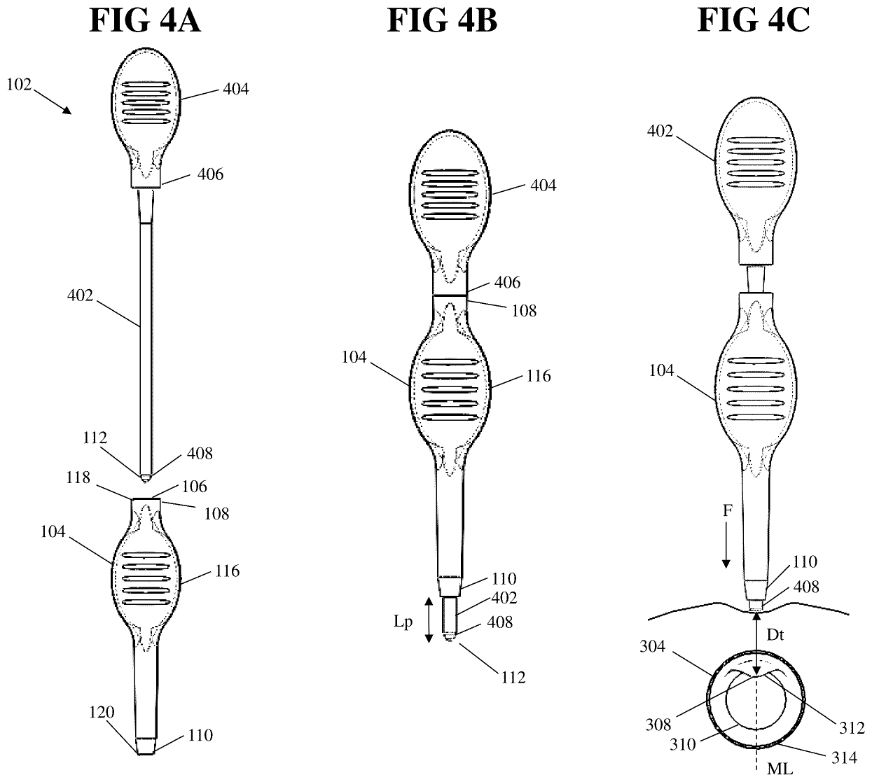

In some implementations, an additional probe (402) may be used. For example, an additional probe may be used to provide a tissue indenting feature (112) that is separate from the locating tip (110). For example, the locating tip (110) may include a guide lumen (106) such that if the tissue indenting feature (112) is a part of the guide (104), it cannot be smaller than the guide lumen (106). In FIGS. 4A-4C, an additional probe (402) is shown with the guide (104). The probe (402) can include its own probe finger interface (404) and probe hard stop (406) that is larger than the diameter of the guide lumen (106) that engages or interacts with the depth controlling stop (108) of the guide (104). The probe (402) further includes a probe blunt end (408) which forms a tissue indenting feature (112). When the probe (402) is inserted into the guide (104), the probe blunt end (408) sticks past the guide distal end (120) of the guide (104) by a probe length Lp. When pressed against the anterior surface (302) of a trachea (304), the probe blunt end (408) acts as a tissue indenting feature (112) that creates a trachea indentation (308) which serves as a visual indicator (312). Beneficially, the probe blunt end (408) may create a more defined trachea indentation (308) by being a smaller tip than permitted by the locating tip (110) of the guide (104) due to the guide lumen (106). After the location of the locating tip (110) is visualized using this method, the probe (402) can be removed, and the needle (202) and cannula (208) can be inserted.

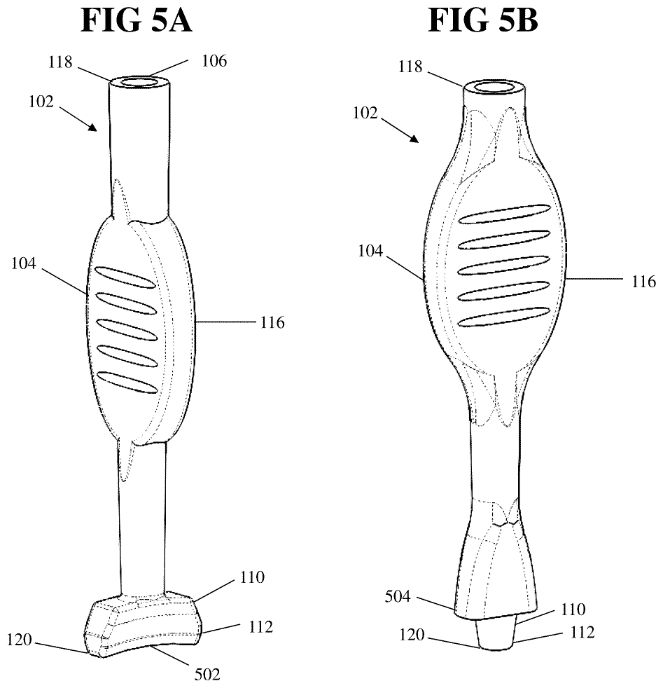

In FIGS. 5A-5B, alterative implementations of the guide (104) are shown. In FIG. 5A, the locating tip (110) further includes a ring positioning feature (502) that can assist in accurately placing the locating tip (110) in the space between the tracheal rings. In one implementation, the ring positioning feature (502) is a rectangular boss with a curved underside that matches the curvature of the trachea (304). In this manner, it can fit within the grove between two tracheal rings. This may beneficially assist in the user being able to feel the tracheal rings and aligning the guide (104) relative to the tracheal lumen (310). The width of the ring positioning feature (502) that fits within the tracheal rings can be from and including about 0.050 inches (1.27 mm) to and including about 0.250 inches (6.35 mm) or about 0.120 inches (3.05 mm). The length can be from an including about 0.100 inches (2.54 mm) to and including about 1.000 inch (25.4 mm) or about 0.500 inches (12.7 mm). The curved underside can have a radius from and including about 0.100 inches (2.54 mm) to and including about 10.000 inches (254 mm) or about 1.100 inches (27.94 mm). In some implementations the underside of the ring positioning feature can be flat. In FIG. 5B, the guide (104) includes a wide portion (504) that can assist in preventing the tissue indenting feature (112) from penetrating the tissue too much. This can beneficially allow the tissue indenting feature (112) to remain narrow and therefore provide clearer visualization yet additionally prevent over insertion into the tissue due to the wide portion (504). The wide portion (504) can have a width from and including about 0.050 inches (1.27 mm) to and including about 0.500 inches (12.7 mm) or about 0.180 inches (4.57 mm). The length of the wide portion (504) can be from and including about 0.050 inches (1.27 mm) to and including about 1.00 inches (2.54 mm) or about 0.350 inches (8.89 mm). The locating tip (110) can extend from the wide portion (504) a length from and including about 0.010 inches (0.25 mm) to and including about 0.400 inches (10.16 mm) or about 0.150 inches (3.81 mm). In some implementations, the wide portion (504) may be circular, elliptical, polygonal, or any other suitable shape.

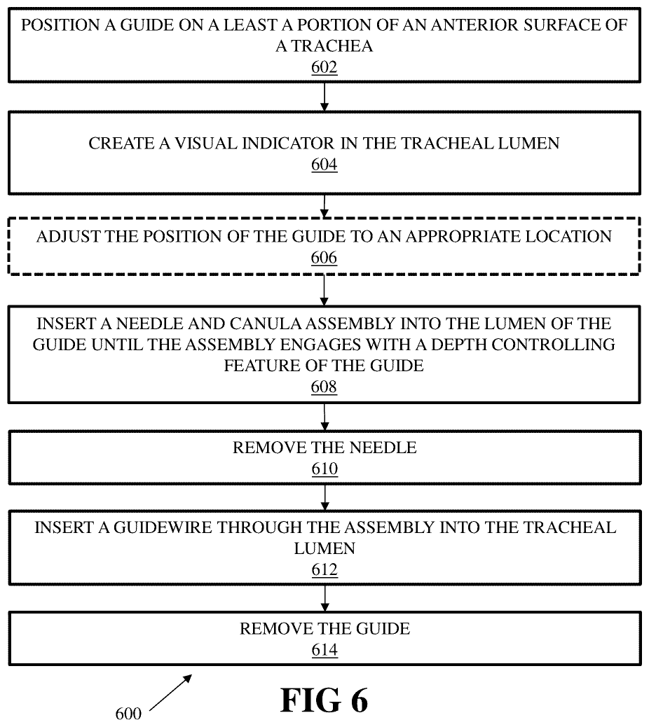

In FIG. 6, a method (600) of performing a tracheostomy or placing a needle and cannula into a patient's trachea is shown. In some implementations, the skin anterior to the trachea may be cut away to expose the anterior surface of the trachea, and a bronchoscope or other visualization element can be placed into the trachea. At block (602), the method includes positioning a guide on at least a portion of an anterior surface of a trachea. For example, the guide may be positioned between adjacent tracheal rings. In some implementations, the guide is positioned between the first and second tracheal rings. In some implementations, the guide is positioned between the second and third tracheal rings. In some implementations, the guide is positioned on a midline of the trachea extending from a dorsal side to a ventral side of the tracheal lumen.

At block (604), the method includes creating a visual indicator within the tracheal lumen, for example to identify a position of the guide relative to the trachea. In some implementations, the visual indicator may include an indentation into the tracheal lumen caused by depressing the guide or a probe into the trachea. In some implementations, the visual indicator may include an illuminated tissue, for example within the tracheal lumen, caused by a light source engaged with or coupled to the guide. In some implementations, the visual indicator may include a sharp protrusion engaged with or coupled to the guide. Although a few visual indicators are described, any other visual indication of the position of the guide may be used.

At block (606), the method optionally (shown by dashed lines) includes adjusting a position of the guide until the visual indicator is in the desired location. For example, the guide may be moved, and force or pressure applied again to create a new visual indicator.

At block (608), the method includes inserting a needle and cannula assembly through the guide lumen of the guide until the assembly engages or interacts with a depth controlling stop of the guide. At block (610), the method includes removing the needle from the lumen of the cannula. At block (612), the method includes inserting a guidewire through the cannula and into the trachea. At block (614), the method includes removing the guide and/or cannula. With the guidewire in place, typical procedures for placing a tracheostomy tube may be performed such as dilating the hole with one or more dilators over the guidewire.

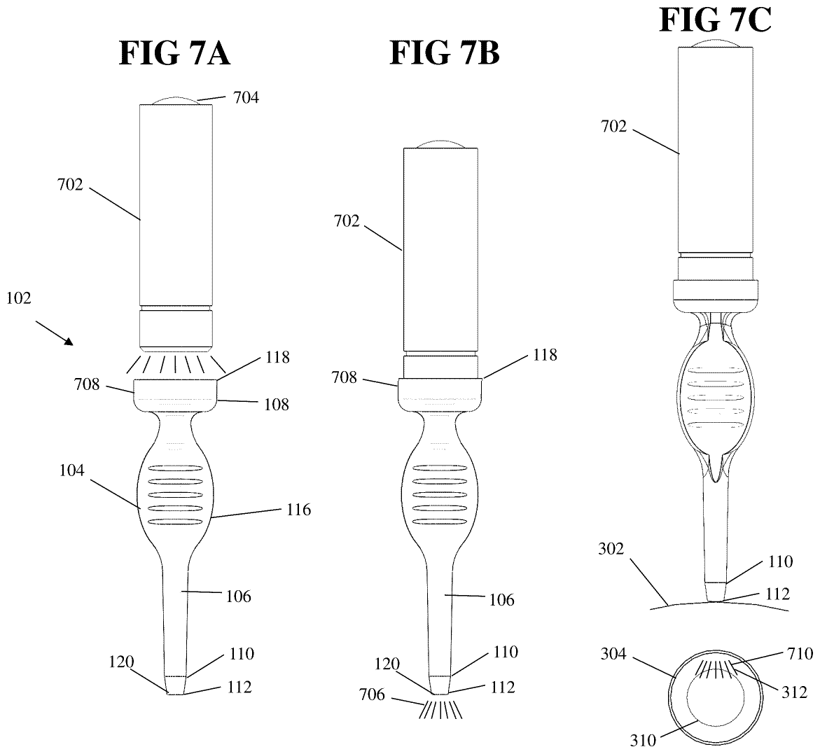

In FIGS. 7A-7C, an implementation of the device (102) with a light source (702) is shown. As shown in FIG. 7A, the light source (702) can be a separate assembly that can be engaged with or coupled to the guide (104). For example, engagement or coupling between the light source (702) and the guide (104) may be a press fit, a bayonet connection, a latched connection, a Luer lock connection, or any other suitable connection. The light source (702) can be any number of light generating assemblies such as a flashlight, a laser, a button light, or any other suitable configuration. The light source (702) may include a separate input element (704) as shown or may be configured to automatically turn on when connected to the guide (104). Light (706) is generated by the light source (702) such that it can be directed into the guide (104). The light (706) may be of any color or wavelength and brightness. For example, a light (706) having a white tone may be used. Alternatively, a light (706), such as a red laser light, may be used and include a high intensity. The wavelength of the light (706) may be from and including about 300 nm to and including about 700 nm. In some implementations, the light (706) may incorporate multiple wavelengths such as in the case of white light. In some implementations, light outside the visible spectrum may be used. The brightness of the light (706) may be from and including about 1 lumen to and including about 10,000 lumens or from and including about 20 lumens to and including about 1,000 lumens, or about 100 lumens. The light may be coherent, or columnated, or scattered depending on the light source (702) and material which the light (706) is transmitted through. In FIG. 7B, the light source (702) is engaged or coupled to the guide (104) at the alignment feature (708). The alignment feature (708) can be a cup that interacts with the outer diameter of the light source (702) or may be any number of other features. For example, in some implementations, the light source (702) and guide (104) can be attached so that they remain together with an interlocking feature that holds them together. For example, the connection may be a bayonet connection, a threaded connection, a magnetic connection, an interference fit, a latched connection, or any number of other means of connecting the components. As shown, the light (706) is projected to the bottom of the guide (104). This may be accomplished by illuminating through the guide lumen (106). The inner surface of the guide lumen (106) can be configured to improve transmission of light such as through plating or electropolishing or any other method. Alternatively, the light (706) may be transmitted through the body of the guide (104) using one or more light pipes (808) as will be described in greater detail below. In FIG. 7C, the device (102) is placed onto, proximate to, or in contact with at least a portion of an anterior surface (302) of a trachea (304) of a patient. The tracheal lumen (310) is shown, and an illuminated tissue (710) is shown. The illuminated tissue (710) can be a visual indicator (312) to the user of the position of the locating tip (110). The user can adjust the location of the guide (104) on the anterior surface of the trachea accordingly so that the locating tip (110) is in an appropriate position. For example, the appropriate position may be the guide aligning with a midline of the trachea (as shown in FIG. 4C), positioning the guide between the first and second tracheal rings, positioning the guide between the second and third tracheal rings, or any at positions desired by the user due to specific anatomy and or other constraints of the patient. The light source (702) can then be removed from the guide (104), and the needle (202) and cannula (208) can be placed through the guide lumen (106) of the guide (104), as described elsewhere herein. In some implementations, the illuminated tissue (710) can be used in conjunction with a trachea indentation (308) to provide a visual indicator (312) to the user.

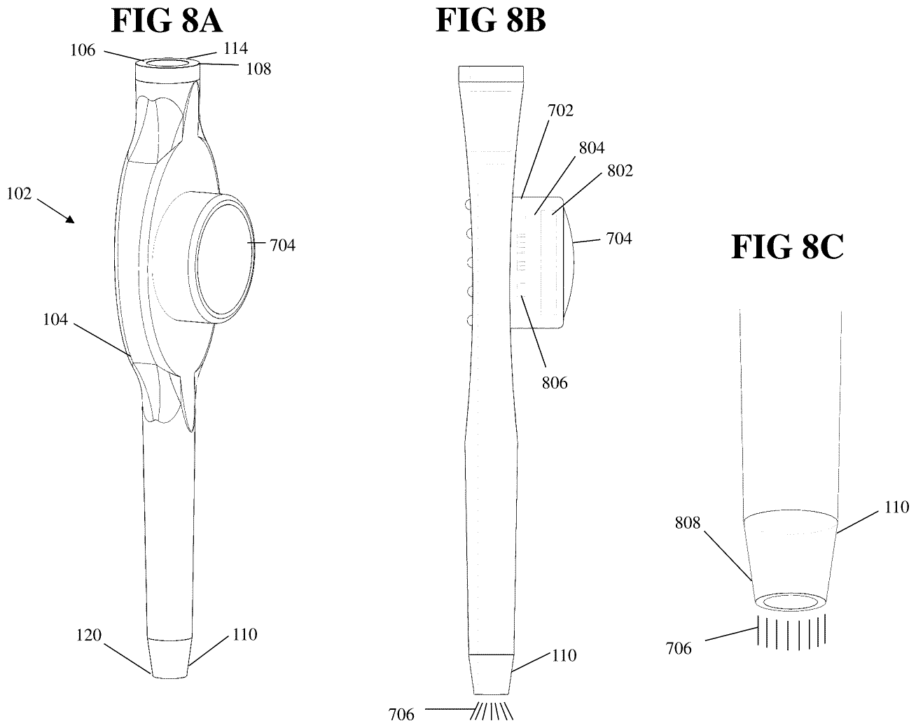

In FIG. 8A, the device 102 is shown with a light source (702) integrated into the guide (104). For example, the light source (702) may include an input element (704), for example a button, slider, toggle switch, etc., on the guide (104), for example on a side of the guide, and may be positioned such that can be easily turned on by a user while positioning the guide (104). An advantage of such a configuration may be that the user can keep the light source (702) illuminated while the needle (202) and cannula (208) are advanced through the guide lumen (106). This allows for constant or substantially constant visual indicator for the user inside the trachea, or in at least a portion of an interior of the trachea, indicating the position of the locating tip (110). In such a configuration, the illuminated tissue (710) may be sufficient as a visual indicator (312) and an indenting feature (112) may not be used. In other configurations, an indenting feature (112) may be still included to provide additional visual indicators (312) of the position of the locating tip (110). In FIG. 8B the light (706) is shown emitting from the guide distal end (120) of the guide (104). The light (706) may be emitted through the guide lumen (106) or may be alternatively emitted through features of the guide (104). For example, the light may be emitted through the locating tip (110), a ring positioning feature (502), a wide portion (504), etc. In FIG. 8C, the light (706) is shown emitted through the locating tip (110) of the guide (104). The guide (104) may therefore include a light pipe (808) that transmits the light (706) from the light source (702) through plastic or other material within the guide (104) rather than through the guide lumen (106). As shown, the body of the guide (104) may become the light pipe (808), or a separate component can be used as a light pipe (808). In some implementations, the light source (702) can be automatically activated when the locating tip (110) of the guide (104) depresses a tissue.

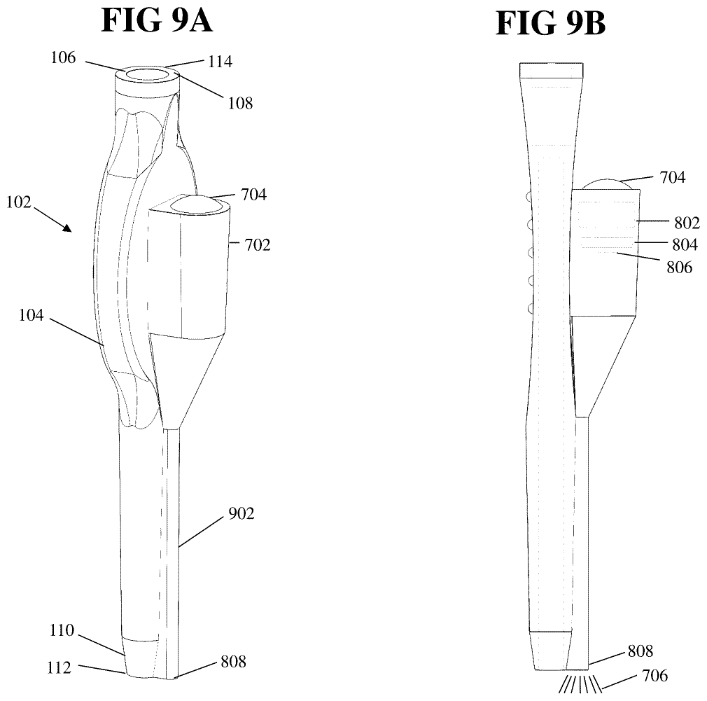

In FIG. 9A, another implementation of the device (102) is shown with a light source (702). The guide (104) includes a separate light channel (902) that is separate from the guide lumen (106). The light channel (902) may be a light pipe (808) as shown or may be a separate hole in the guide lumen (106). In FIG. 9B, the device (102) is further shown with a battery (802), a circuit board (804), and a light source (806), for example a light emitting diode, a laser, or similar. The light (706) comes from the light source (806) and is transmitted to the guide distal end (120) of the guide (104) through a light channel (902). In the implementation shown, the light channel (902) is a plastic light pipe (808), but any number of other configurations are contemplated including through lumens, fiber optics, and the like. The light (706) may be emitted close enough to the locating tip (110) such that no substantial correction may be made by the user in identifying the position of the locating tip (110). In other implementations, the distance between the locating tip (110) and illuminated tissue (710) can be corrected for by the user.

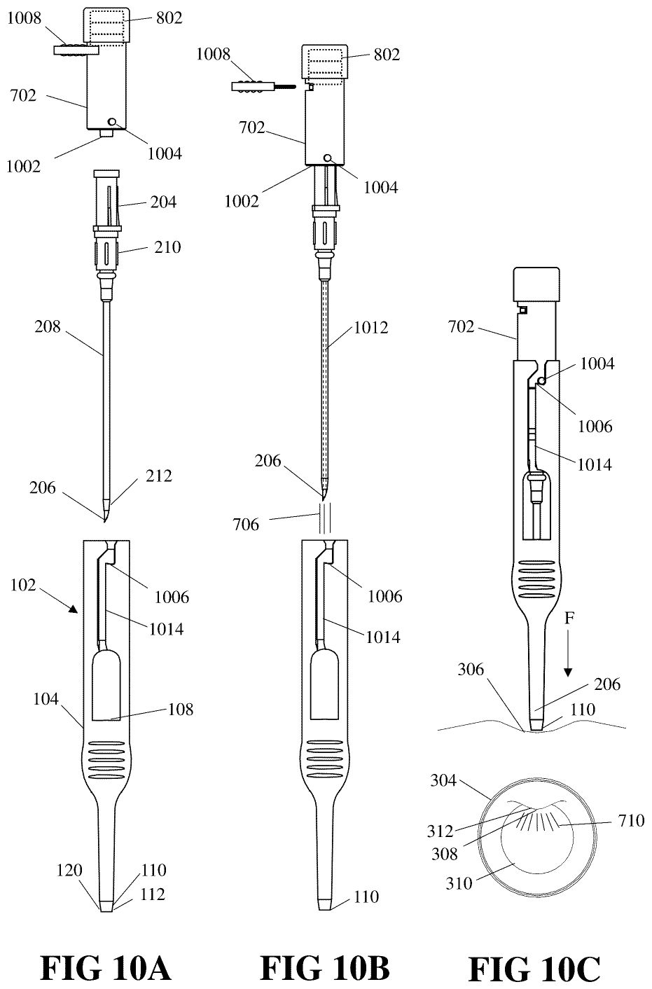

In FIGS. 10A, another implementation of the device (102) is shown with a light source (702). The light source (702) includes a battery separator (1008) and a light connector (1002). When illuminated, the light (706) is emitted from the light connector (1002) and the light connector (1002) is configured to connect to the needle hub (204) and shine a light (706) through the lumen of the needle (202). The battery separator (1008) is a component that physically blocks the electrical connection within the light source (702) when in the position shown such that the light source (702) is not activated. For example, battery separator (1008) can be a piece of non-conducting paper or plastic that separates the batteries from each other or separates a battery from an electrical connection within the light source (702).

In FIG. 10B, the battery separator (1008) is removed, and the light source (702) is turned on by the established electrical path between the power source, for example battery (802), and the light source (702). In some implementations, the light source (702) can include an input element (704) that is activated or selected by a user to activate the light source (702) in replacement of or in addition to the battery separator (1008). The light source (702) and needle (202) are connected through the light connector (1002) and the light (706) is transmitted through the needle lumen (1012) to the needle tip (206). The light (706) may be collimated laser light or diffuse white light or any other suitable type of light. The light connector (1002) can be a Luer lock, a threaded connection, a bayonet connection, or any other type of interlocking connection. In some implementations, the light source (702) and needle (202) are fixedly connected and not intended for the user to separate them. In such an implementation, the light connector (1002) is any number of elements that connects the light source (702) to the needle (202) and allow transmission of the light (706) through the lumen of the needle (202) and to the needle tip (206). A Luer lock connection may be well suited for the light connector (1002) since many needles (202) have a Luer lock connection already and these types of connectors are familiar to medical users. In other implementations, the light connector (1002) may be a bayonet connection, a tapered connection, a threaded connection, etc.

In FIG. 10C, the light source (702) and needle (202) are inserted into the guide lumen (106) of the guide (104). As described herein, the guide (104) can be positioned above a tracheal lumen (310) of patient. The guide (104) can be depressed with downward force F to create an external indentation (306), for example an indentation in the tissue or dimple in the tissue, and a trachea indentation (308) or visual indicator (312) in a lumen of the trachea or an interior portion of the trachea. In addition, the light (706) shines through the needle (202) and guide (104) and onto an external surface of the trachea. The user can then observe the visual indicator (312) of the illuminated tissued within a lumen of the trachea (304) and can optionally adjust the position of the guide (104) accordingly until the desired position is obtained (e.g., based on a midline of the trachea, rings of the trachea, etc.). As shown, the device (102) can create multiple visual indicators (312) to provide additional visual confirmation. An advantage of having the light (706) shine through the lumen of the needle (202) is that it can illuminate tissue throughout the procedure so the user can confirm location at any time. The light source (702) includes a positioning pin (1004) which is embodied as a detent or circular boss, and the guide (104) includes a retraction hard stop (1006). The positioning pin (1004) engages or interacts with the retraction hard stop (1006) such that the movement or travel of the needle (202) is limited, and the needle tip (206) remains within the guide (104). This allows the user to maneuver the locating tip (110) and depress on at least a portion of an anterior surface (302) without the needle tip (206) protruding and inserting into at least a portion of the anterior surface (302). The user can then confirm one or more visual indicators (312) within the tracheal lumen (310) and manipulate the guide (104) and locating tip (110) to the appropriate position (e.g., based on a midline of the trachea, rings of the trachea, etc.). Any number of other engagement or coupling features between the needle (202), cannula (208), light source (702), and the guide (104) are contemplated. In some implementations, the needle hub (204) or the cannula hub (210) can include the positioning pin (1004). In other implementations, the guide (104) can include one or more detents that retain the slidable assembly at fixed locations. In still other implementations, the guide (104) and slidable assembly can have an interference fit to hold it in a retracted position. Any number of other means of engagement or coupling are contemplated.

In FIG. 10D, the light source (702) is rotated such that the positioning pin (1004) can move or travel past the retraction hard stop (1006) and into a guide track (1014) such that the needle (202) can be advanced with the needle tip (206) inserted into the tracheal lumen (310). During insertion of the needle (202) into the tracheal wall, the tissue can remain illuminated providing further visual indicators (312) of the position and depth of the needle tip (206) in the tracheal wall. The cannula hub (210) can press into the depth controlling stop (108) as shown. In other implementations, the depth controlling stop (108) can engage or interact with the positioning pin (1004) to limit the axial movement or travel of the needle (202). For example, the guide track (1014) can form a depth controlling stop (108) such that the guide track (1014) ends and the downward movement, or advancement of the positioning pin (1004) is prevented at the depth controlling stop (108).

In FIG. 10E, the light source (702) and needle (202) are removed, and the cannula (208) is left in place within the tracheal lumen (310). In FIG. 10F, a guidewire (1010) is inserted into the cannula (208) and advanced into the tracheal lumen (310). In some implementations, the guide track (1014) used for the positioning pin (1004) serves as a slot which enables the guidewire (1010) to be directly inserted into the cannula hub (210) and then moved concentrically into the guide (104). In FIG. 10G, the guide (104) is removed, and the guidewire (1010) remains in the tracheal lumen (310). The guidewire (1010) can be used for the insertion of one or more dilators to increase the size of the created hole.

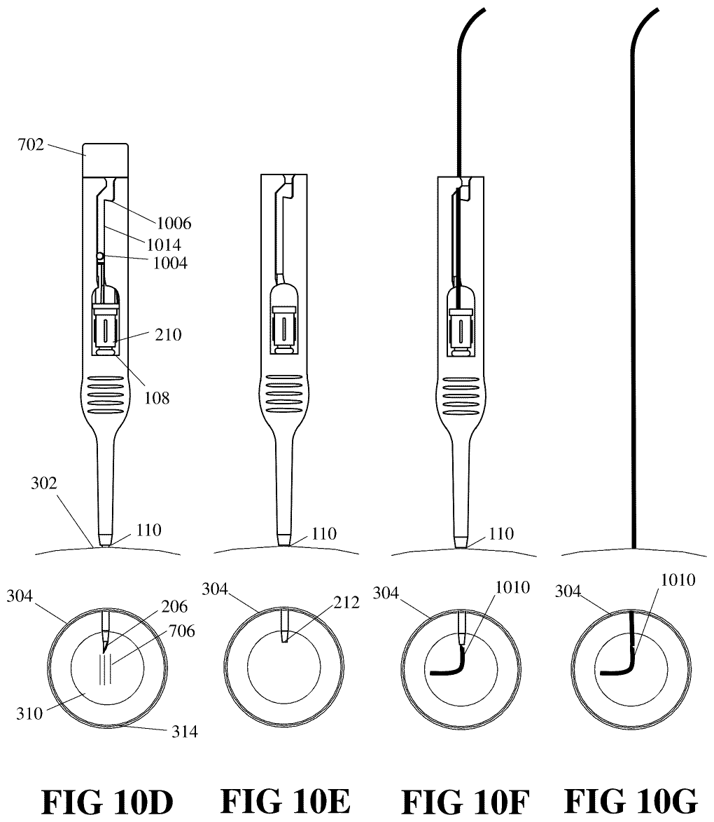

In FIG. 11A, an implantation of the device (102) is shown with a sharp protrusion (1202) attached at the locating tip (110). The sharp protrusion (1202) can include a small gauge needle with an outer diameter from and including about 0.004 inches (0.10 mm) to and including about 0.050 inches (1.27 mm) or about 0.014 inches (0.36 mm). The length of the sharp protrusion (1202) can be from and including about 0.25 inches (6.35 mm) to and including about 3.00 inches (76.2 mm) or about 1.00 inch (25.4 mm). The sharp protrusion (1202) includes a sharpened tip with a hollow lumen such as a needle. In other implementations, the sharp protrusion (1202) can be a non-hollow element and may also not require a sharpened tip if the diameter is sufficiently small. In some implementations, the sharp protrusion (1202) can be detachable or moveable relative to the guide (104). As an example, the sharp protrusion (1202) can slide axially along the length of the guide (104) such that it can be inserted and removed as desired by the user. In other implementations, the sharp protrusion (1202) can be small gauge needle that is attached to the guide (104) using a Luer connection.

In FIG. 11B, the guide (104) is placed onto, proximate to, or in contact with at least a portion of the anterior surface (302) and the sharp protrusion (1202) extends into the tracheal lumen (310). The sharp protrusion (1202) then provides a visual indicator (sharp protrusion 1202 extending into the tracheal lumen 3 (310)) to the user of the location of the locating tip (110) and where the needle (202) will enter if advanced through the guide lumen (106). Beneficially, a sharp protrusion (1202) can cause insignificant or minimal damage to the surrounding tissue structures. Optionally, the user can then remove and adjust the position of the guide (104) accordingly until satisfied with the location of the visual indicator. Additionally, the sharp protrusion (1202) can assist in maintaining the position of the guide (104) on at least a portion of the anterior surface (302) by securing it to the tissue. This can be particularly useful such that the locating tip (110) does not slip on at least a portion of the anterior surface (302). In some implementations, additional elements may be combined such as a light source (702) to create multiple visual indicators (312).

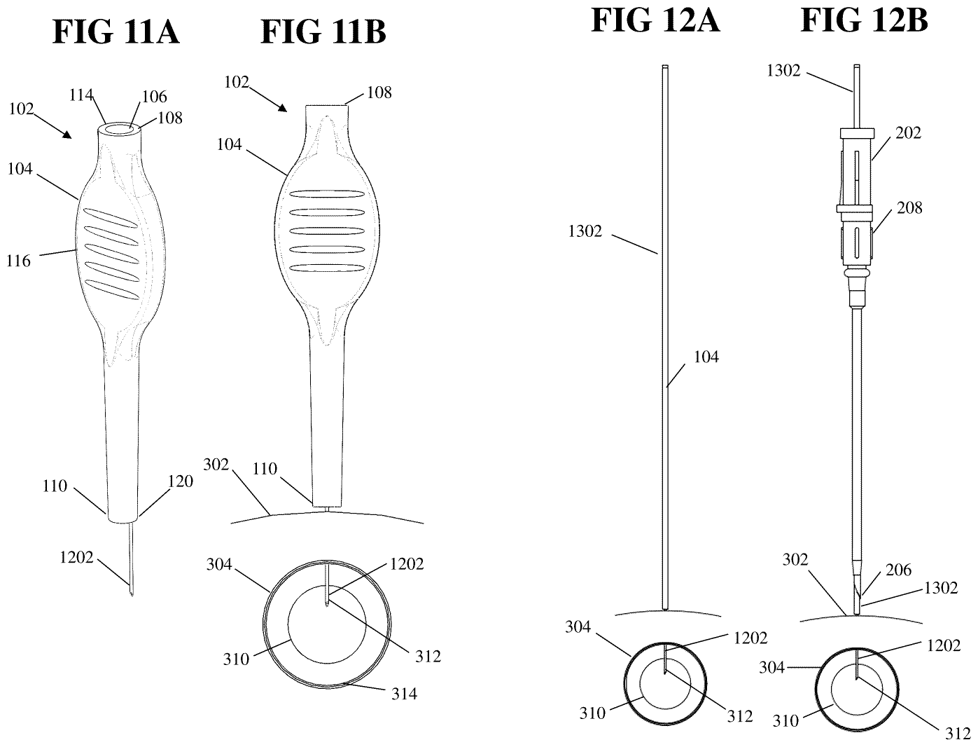

In FIG. 12A, an implementation of the device (102) is shown with a sharp protrusion (1202) on a stylet (1302). The stylet (1302) can have a larger diameter than the sharp protrusion (1202) such that the depth of the sharp protrusion (1202) is controlled since the stylet (1302) cannot be easily inserted into the trachea (304). The stylet (1302) diameter can be from and including about 0.040 inches (1.01 mm) to and including about 0.200 inches (5.08 mm) or about 0.060 inches (1.52 mm). In some implementations, the stylet (1302) diameter fits within the needle lumen (1012). The stylet (1302) length can be from and including about 0.100 inches (2.54 mm) to and including about 20 inches (508 mm) or about 8 inches (203.2 mm). The sharp protrusion is shown inserted into the trachea (304) and forms a visual indicator (312). Optionally, the user can remove and reinsert the sharp protrusion (1202) to adjust the position of the visual indicator (312). As will be shown, the stylet (1302) forms a guide (104), that is intralumenal, for the needle (202) and cannula (208).

In FIG. 12B, the needle (202) and cannula (208) are advanced over the stylet (1302). The stylet (1302) serves as a guide (104) for the needle (202) so that the needle (202) is advanced to the visual indicator (312) created by the sharp protrusion (1202).

In some implementations, an input element (704) can be incorporated into the light connector (1002) such that the light source (702) is activated when it is connected to the needle hub (204). In some implementations, the needle (202) can be placed into the trachea using the light source (702) but without the guide (104).

In some implementations, the probe (402) shown in FIGS. 4A-4C may include a light source (702) and may include one or more features at the probe blunt end (408) to emit light that is generated by the light source (702). In this manner, the probe (402) with the light source (702) can be used to further identify the position of the locating tip (110) prior to insertion of the needle (202) or cannula (208).

In some implementations, the guide lumen (106) can be a cylindrical hole that extends from the guide proximal end (118) to guide distal end (120) of the guide (104). In other implementations, the guide lumen (106) can form a partial lumen such as a semi-circular cross-section. This beneficially could allow the guide (104) to be removed from the cannula (208) once the cannula (208) is inserted in the tracheal lumen (310).

In some implementations, the guide (104) can have a variety of shapes. For example, rather than being a long tube with a finger interface, the guide (104) can be shaped more like an arc that fits onto at least a portion of the anterior surface (302) of the trachea (304) or neck or rests at least partially circumferentially along a dorsal portion of the trachea (304). Beneficially, in such an implementation, the guide (104) can rest with increased stability against the tissue, such that the user can hold the guide (104) more loosely. The arc can match the contour of the tissue and can therefore assist in providing indication of the midline of the trachea.

In some implementations, the device (102) can include measurement and indicator elements to assist in maintaining proper orientation of the guide (104). For example, the guide (104) can include a bubble level or level indicator that indicates the orthogonal orientation of the guide (104). In other implementations, the guide (104), cannula (208), or needle (202) can include markings that indicate the depth of penetration relative to one another.

In some implementations, the cannula engagement feature (114) may include a screw mechanism that controls the insertion depth of the needle (202) and cannula (208) into the tracheal lumen (310). For example, the cannula engagement feature (114) can be a female thread while the cannula hub (210) can have a corresponding external thread. In this manner, the needle (202) and cannula (208) can be linearly advanced by screwing the cannula (208) into the guide (104). Beneficially, this may allow the user finer control of the depth of placement of the needle (202) and cannula (208) into the tracheal lumen (310). This may be particularly useful where the tissue depth Dt is inconsistent and therefore a variable depth controlling stop (108) such as this would useful. Some implementations of the device (102) may include indicators for when the needle (202) is within the tracheal lumen (310). For example, the device (102) can use air pressure checks to determine if the needle tip (206) is within tissue or has entered the tracheal lumen (310). Such an implementation can allow the user to depress the needle (202) and cannula (208) until they have confirmed it is within the tracheal lumen (310).

Much description has been given to tracheostomy procedures but that is not intended to limit the scope or use of the device (102) to such procedures. The device (102) can have a variety of shapes and sizes serving as a platform for any appropriate procedure. For example, the device (102) can be used for tracheostomies including percutaneous tracheostomies and open tracheostomies. Additionally, the device (102) can be used for an emergency cricothyrotomy. Additionally, the device (102) can be used for laparoscopic surgery and the placement of trocars and instruments within the abdominal cavity. Light sources and indentation methods can be used for visualizing the placement of trocars prior to insertion to ensure proper location. Additionally, the device (102) can be used for the placement of Percutaneous Endoscopic Gastrostomy (PEG) tubes, where a guide is used to advance a needle to the stomach of a patient in preparation for placement of a PEG tube.

The names and labels applied to the various components and parts may not be considered limiting to the scope of the invented device and method.

Although implementations of various methods and devices are described herein in detail with reference to various versions, it can be appreciated that other versions, implementations, methods of use, and combinations thereof are also possible. Therefore, the spirit and scope of the appended claims should not be limited to the description of the implementations contained herein. References in the specification to “one implementation,” “an implementation,” “an illustrative implementation,” “some implementations,” etc., indicate that the implementation described may include a particular feature, structure, or characteristic, but every implementation may or may not necessarily include that particular feature, structure, or characteristic. Moreover, such phrases are not necessarily referring to the same implementation. Further, when a particular feature, structure, or characteristic is described in connection with an implementation, it is submitted that it is within the knowledge of one skilled in the art to effect such feature, structure, or characteristic in connection with other implementations whether or not explicitly described.

As used in the description and claims, the singular form “a”, “an” and “the” include both singular and plural references unless the context clearly dictates otherwise. For example, the term “guide” may include, and is contemplated to include, a plurality of guides. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Examples

Example 1. A method of performing a tracheostomy procedure, comprising positioning a locating tip of a guide proximate to or in contact with at least a portion of an anterior surface of a trachea, the guide and the locating tip having a guide lumen extending therethrough; creating a visual indicator within the trachea; and inserting a needle through the guide lumen and into the trachea according to the visual indicator.

Example 2. The method of any one of the preceding examples, but particularly Example 1, wherein the creating comprises depressing the locating tip of the guide into the at least a portion of the anterior surface of the trachea to indent the at least a portion of the anterior surface of the trachea.

Example 3. The method of any one of the preceding examples, but particularly Example 1, wherein the creating comprises illuminating, using a light source coupled to the guide, at least a portion of an interior surface of the trachea.

Example 4. The method of any one of the preceding examples, but particularly Example 3, further comprising coupling the light source to the guide.

Example 5. The method of any one of the preceding examples, but particularly Example 1, wherein the creating comprises inserting a sharp protrusion through the guide lumen and into a lumen of the trachea.

Example 6. The method of any one of the preceding examples, but particularly Example 1, wherein the inserting comprises inserting the needle into a cannula and inserting the cannula into the guide lumen.

Example 7. The method of any one of the preceding examples, but particularly Example 1, further comprising visualizing the visual indicator within the trachea with a bronchoscope.

Example 8. The method of any one of the preceding examples, but particularly Example 1, further comprising adjusting a position of the locating tip according to the visual indicator.

Example 9. The method of any one of the preceding examples, but particularly Example 1, wherein the inserting further comprising rotating the needle to advance the needle through the guide lumen.

Example 10. The method of any one of the preceding examples, but particularly Example 1, wherein the positioning comprises positioning the locating tip of the guide between a second tracheal ring and a third tracheal ring.

Example 11. The method of any one of the preceding examples, but particularly Example 1, wherein the inserting is limited by interaction between a depth controlling stop on the guide and a cannula hub.

Example 12. The method of any one of the preceding examples, but particularly Example 1, wherein the creating comprises: depressing the locating tip of the guide into the at least a portion of the anterior surface of the trachea to indent the at least a portion of the anterior surface of the trachea, and illuminating, using a light source coupled to the guide, at least a portion of an interior surface of the trachea.

Example 13. The method of any one of the preceding examples, but particularly Example 1, wherein the tracheostomy is a percutaneous tracheostomy.

Example 14. The method of any one of the preceding examples, but particularly Example 1, wherein the tracheostomy is an open tracheostomy.

Example 15. A device for performing a tracheostomy procedure, comprising: a guide defining a guide lumen and comprising; a locating tip having a tissue indenting feature, wherein the guide lumen extends through the locating tip; and a depth controlling stop for controlling a depth of insertion of a needle through the guide lumen and into a lumen of a trachea.

Example 16. The device of any one of the preceding examples, but particularly Example 15, further comprising the needle.

Example 17. The device of any one of the preceding examples, but particularly Example 16, further comprising a light source that is attached to the needle and is configured to shine light through a lumen of the needle.

Example 18. The device of any one of the preceding examples, but particularly Example 16, further comprising a cannula, wherein the needle is configured to be inserted through the cannula and the cannula is configured to be inserted through the guide lumen.

Example 19. The device of any one of the preceding examples, but particularly Example 16, wherein the guide further comprises a hard stop and the needle further comprises a pin that is configured to interact with the hard stop to prevent movement of the needle.

Example 20. The device of any one of the preceding examples, but particularly Example 19, wherein the guide defines a track, wherein the needle is configured to be rotated to allow the pin to travel a length of the track to advance the needle into a tissue of the trachea.

Example 21. The device of any one of the preceding examples, but particularly Example 15, wherein the tissue indenting feature is configured to depress at least a portion of an anterior surface of the trachea to create a visual indicator in at least a portion of the lumen of the trachea.

Example 22. The device of any one of the preceding examples, but particularly Example 15, further comprising a light source integrated into the guide or coupled to the guide, wherein the light source is configured to create a visual indicator comprising illuminated tissue in at least a portion of the lumen of the trachea.

Example 23. The device of any one of the preceding examples, but particularly Example 22, wherein the light source is configured to shine through the guide lumen.

Example 24. The device of any one of the preceding examples, but particularly Example 15, further comprising a finger interface that enables a user to press the tissue indenting feature into at least a portion of an anterior surface of the trachea.

Example 25. The device of any one of the preceding examples, but particularly Example 15, further comprising a sharp protrusion which is configured to create a visual indicator by entering into the lumen of the trachea when the locating tip is against an anterior surface of the trachea.

Example 26. A method of performing an emergency cricothyrotomy procedure, comprising positioning a locating tip of a guide proximate to or in contact with at least a portion of an anterior surface of a trachea, the guide and the locating tip having a guide lumen extending therethrough; creating a visual indicator within the trachea; and inserting a needle through the guide lumen and into the trachea according to the visual indicator.

Example 27. The method of any one of the preceding examples, but particularly example 26, wherein the positioning comprises positioning the locating tip of the guide between a thyroid cartilage and a cricoid cartilage.

Example 28. A method of performing a laparoscopic procedure, comprising positioning a locating tip of a guide proximate to or in contact with at least a portion of an anterior surface of an abdominal region, the guide and the locating tip having a guide lumen extending therethrough; creating a visual indicator within the abdominal region; and inserting a needle through the guide lumen and into the abdominal region according to the visual indicator.

Example 29. A method for positioning a percutaneous endoscopic gastrostomy (PEG) tube, comprising positioning a locating tip of a guide proximate to or in contact with at least a portion of an abdominal region overlying a stomach, the guide and the locating tip having a guide lumen extending therethrough; creating a visual indicator within the abdominal region; and inserting a needle through the guide lumen and into a stomach according to the visual indicator.

1. A method of performing a tracheostomy procedure, comprising positioning a locating tip of a guide proximate to or in contact with at least an external portion of an anterior surface of a trachea, the guide and the locating tip having a guide lumen extending therethrough;
creating a visual indicator within the trachea to identify an entry point of a needle from the at least an external portion of the anterior surface of the trachea and into a lumen of the trachea, wherein the visual indicator is transmitted from the guide positioned external to the trachea and into an interior portion of the trachea; and
inserting a needle through the guide lumen external to the trachea and into the trachea according to the visual indicator.

2. The method of claim 1, comprising depressing the locating tip of the guide into the at least an external portion of the anterior surface of the trachea to indent the at least an external portion of the anterior surface of the trachea to cause an indentation in the lumen of the trachea.

3. The method of claim 1, wherein the creating comprises illuminating, using a light source coupled to the needle, at least a portion of an interior surface of the trachea to guide the needle to the visually indicated location in the lumen of the trachea.

4. (canceled)

5. The method of claim 1, wherein the creating comprises inserting a sharp protrusion through the guide lumen and into the lumen of the trachea.

6. The method of claim 1, wherein the inserting comprises inserting the needle into a cannula and inserting the cannula into the guide lumen.

7. The method of claim 1, further comprising visualizing the visual indicator within the trachea with a bronchoscope.

8. The method of claim 1, further comprising adjusting a position of the locating tip according to the visual indicator.

9. (canceled)

10. The method of claim 1, wherein the positioning comprises positioning the locating tip of the guide between a second tracheal ring and a third tracheal ring.

11. The method of claim 1, wherein the inserting is limited by interaction between a depth controlling stop on the guide and a cannula hub.

12. The method of claim 1, wherein the creating comprises: depressing the locating tip of the guide into the at least an external portion of the anterior surface of the trachea to indent the at least an external portion of the anterior surface of the trachea, and illuminating, using a light source coupled to the needle, at least a portion of an interior surface of the lumen of the trachea.

13. A device for performing a tracheostomy procedure, comprising:
a guide defining a guide lumen and comprising;
a locating tip configured for placement on an external portion of a trachea, wherein the guide lumen extends through the locating tip;
a needle; and
a light source attached to the device and configured to create a visual indicator comprising illuminated tissue within at least a portion of a lumen of the trachea.

14. The device of claim 13, further comprising a depth controlling stop for controlling a depth of insertion of the needle through the guide lumen and into the lumen of the trachea.

15. The device of claim 14, wherein the light source that is attached to the needle and is configured to shine light through a lumen of the needle.

16. The device of claim 14, further comprising a cannula, wherein the needle is configured to be inserted through the cannula and the cannula is configured to be inserted through the guide lumen.

17. The device of claim 14, wherein the guide further comprises a hard stop and the needle further comprises a pin that is configured to interact with the hard stop to prevent axial movement of the needle prior to insertion into the lumen of the trachea.

18. The device of claim 17, wherein the guide defines a track, wherein the needle is configured to be rotated to allow the pin to travel a length of the track to advance the needle into a tissue of the trachea.

19. The device of claim 13, further comprising a tissue indenting feature at the locating tip, wherein the tissue indenting feature is configured to depress at least a portion of an anterior surface of the trachea to create a visual indicator in at least a portion of the lumen of the trachea.

20. (canceled)

21. The device of claim 13, wherein the light source is attached to the guide and configured to shine through the guide lumen.

22. The device of claim 13, further comprising a tissue indenting feature at the locating tip; and a finger interface that enables a user to press the tissue indenting feature into at least a portion of an anterior surface of the trachea.

23. The device of claim 13, further comprising a sharp protrusion which is configured to create a visual indicator by entering into the lumen of the trachea when the locating tip is against an anterior surface of the trachea.
